(19) United States
(12) Patent Application Publication
(10) Pub. No.: US 2014/0317821 A1
(43) Pub. Date: Oct. 30, 2014

(54) Protection Garment

(71) Applicant: Marlene Berns, Beverly Hills, CA (US)

(72) Inventor: Marlene Berns, Beverly Hills, CA (US)

(21) Appl. No.: 13/869,807

(22) Filed: Apr. 24, 2013

(51) Int. Cl. A41D 1/06; A61F 6/04

(52) U.S. Cl. CPC A41D 1/06; A61F 6/04
USPC 2/69

(57) ABSTRACT

A garment is presently disclosed. The garment contains a rear panel, and a crotch panel coupled with the rear panel defining a condom coupling member, wherein the rear panel and the crotch panel form a first leg opening and a second leg opening, wherein the condom coupling member is configured to couple a condom with the crotch panel, wherein the crotch panel comprises material configured to prevent STDs that are transmitted through skin-to-skin contact from passing through the crotch panel.

### FIELD

The present invention relates to garments. More particularly, the present invention relates to garment configured to protect against sexually transmitted diseases (STDs).

### BACKGROUND

Male condoms are elongated, cylindrical sheaths of thin latex worn by males during sexual intercourse. A major problem with male condoms is they only cover and protect the male penis against STDs that are transmitted through skin-to-skin contact. STDs that are transmitted through skin-to-skin contact include, for example, herpes, HPV virus, and syphilis.

In view of the limitation in prior art, a need exists for an improved way of protecting males and females against STDs that are transmitted through skin-to-skin contact.

### BRIEF DESCRIPTION OF THE FIGURES

FIG. 1 depicts an embodiment of a garment according to the present disclosure.

FIG. 2 depicts another view of the garment in FIG. 1.

FIG. 3 depicts side, cutaway, view of the garment in FIG. 1.

FIG. 4 depicts another side, cutaway, view of the garment in FIG. 1.

FIG. 5 depicts an embodiment of another garment according to the present disclosure.

FIG. 6 depicts another view of the garment in FIG. 5.

FIG. 7 depicts side, cutaway, view of the garment in FIG. 5.

FIGS. 8-10 depict a fastener according to the present disclosure.

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements, and are not drawn to scale.

### DETAILED DESCRIPTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

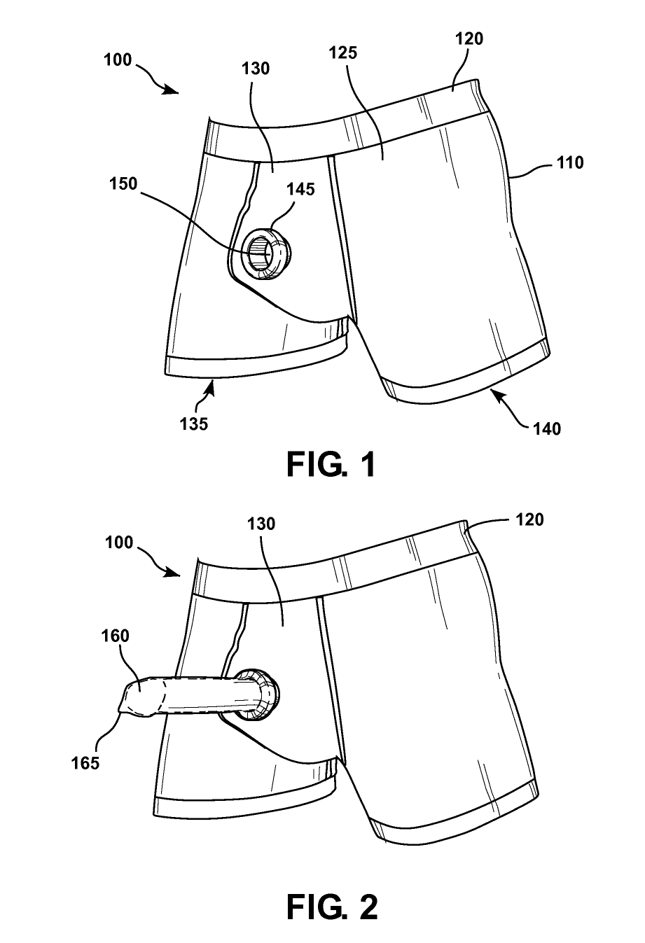

FIG. 1 depicts a garment 100 for a person to wear on their body. The garment 100 can be an undergarment or other garment that is at least partially worn directly against the wearer's skin. In the case of an undergarment, it may have various shapes and styles such as, but not limited to, briefs, shorts, etc. An undergarment covers the body from about the waist down to at least the groin. The undergarment is typically adapted to cover the genitalia of the wearer. Undergarment can further cover the posterior, e.g., gluteus maximus muscles, of the wearer. In one embodiment, the garment 100 extends down the wearer's body no further than onto the wearer's thighs. The garment 100 is usually worn next to the wearer's skin as the first layers of clothing. In one embodiment, the garment 100 is at least partially comprised of a light fabric and snugly fits against the wearer's body.

The garment 100 comprises a garment body 110 supported by a supporting band 120. The supporting band 120 is designed to extend around the wearer's waist. The band 120 includes an elasticized component to hold the undergarment on the waist of the wearer. Examples of an elastic component are thermoplastic polyurethane tape, spandex, Lycra™, certain nylons, rubber, nylon, elastane and lurex elastic, etc. The garment body 110 further comprises a first leg opening 135 and a second leg opening 140.

The garment body 110 includes a rear panel (not shown) to cover the posterior portion of the wearer. The rear panel is joined to at least one front panel 125 to define the body 110. The front panel 125 comprises a crotch panel 130 configured to cover the crotch and genitals of the wearer. In one embodiment, the crotch panel 130 comprises moisture impermeable material designed to prevent STDs that are transmitted through skin-to-skin contact from passing through the crotch panel 130. In one embodiment, the crotch panel 130 comprises latex material coupled with the front panel 125. In another embodiment, the crotch panel 130 is sewn to the front panel 125.

In another embodiment, the front panel 125 comprises moisture impermeable material designed to prevent STDs that are transmitted through skin-to-skin contact from passing through the front panel 125. In one embodiment, the front panel 125 comprises latex material coupled with the crotch panel 130.

In another embodiment, the rear panel comprises moisture impermeable material designed to prevent STDs that are transmitted through skin-to-skin contact from passing through the rear panel. In one embodiment, the rear panel comprises latex material coupled with the front panel 125.

Referring to FIGS. 1 and 2-3, in one embodiment, the crotch panel 130 comprises a condom coupling member 145. The condom coupling member 145 defines an opening 150 sufficient to accommodate the wearer's penis 160. In one embodiment, the condom coupling member 145 comprises latex material. In another embodiment, the condom coupling member 145 is part of the crotch panel 130.

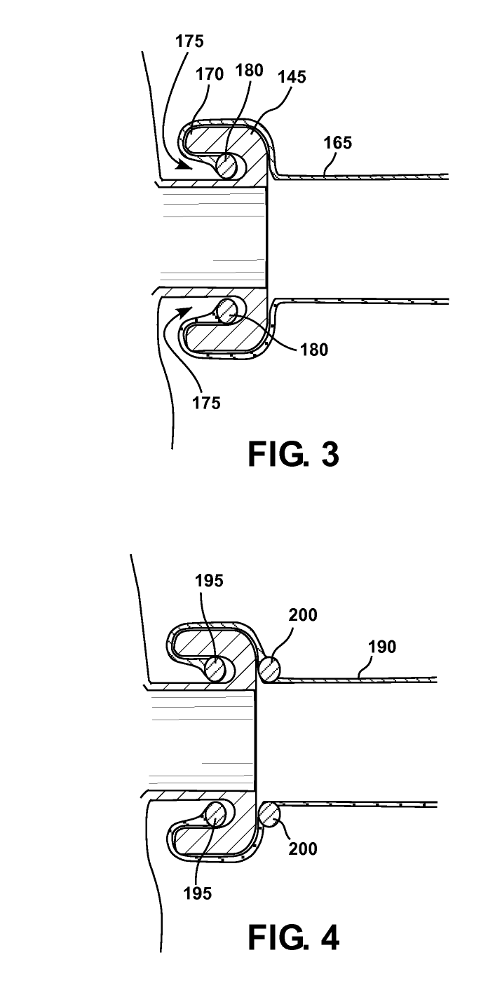

Before intercourse, the wearer of the garment 100 couples a condom 165 to the condom coupling member 145 to protect penis 160 from STDs. Because the crotch panel 130 comprises moisture impermeable material, the crotch panel 130 protects the wearer's crotch area from STDs that are transmitted through skin-to-skin contact. Referring to FIG. 3, in one embodiment, the condom coupling member 145 comprises an outer edge 170 that forms a trench 175 configured to accommodate a rubber band 180 associated with the condom 165. The trench 175 is configured to prevent the condom 165 from slipping off of the condom coupling member 145.

Referring to FIG. 4, in another embodiment, a condom 190 comprises a rubber band 195 and a rubber band 200. In this embodiment, the condom coupling member 145 is configured to couple the condom 190 to the crotch panel 130 as shown in FIG. 4.

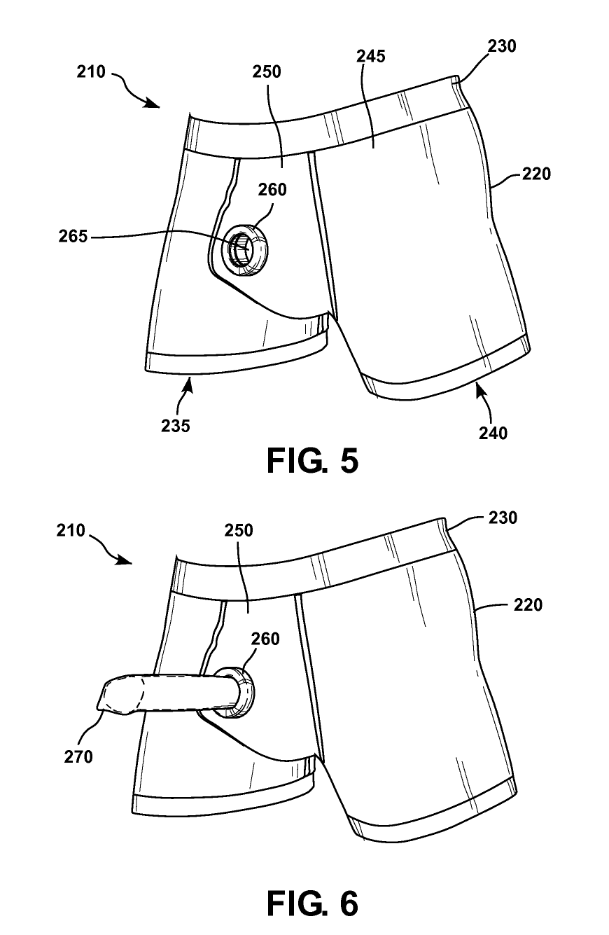

Referring to FIG. 5, a garment 210 is shown according to the present disclosure. The garment 210 can be an undergarment or other garment that is at least partially worn directly against the wearer's skin. In the case of an undergarment, it may have various shapes and styles such as, but not limited to, briefs, shorts, etc. An undergarment covers the body from about the waist down to at least the groin. The undergarment is typically adapted to cover the genitalia of the wearer. Undergarment can further cover the posterior, e.g., gluteus maximus muscles, of the wearer. In one embodiment, the garment 210 extends down the wearer's body no further than onto the wearer's thighs. The garment 210 is usually worn next to the wearer's skin as the first layers of clothing. In one embodiment, the garment 210 is at least partially comprised of a light fabric and snugly fits against the wearer's body.

The garment 210 comprises a garment body 220 supported by a supporting band 230. The supporting band 230 is designed to extend around the wearer's waist. The band 230 includes an elasticized component to hold the undergarment on the waist of the wearer. Examples of an elastic component are thermoplastic polyurethane tape, spandex, Lycra™, certain nylons, rubber, nylon, elastane and lurex elastic, etc. The garment body 220 further comprises a first leg opening 235 and a second leg opening 240.

The garment body 220 includes a rear panel (not shown) to cover the posterior portion of the wearer. The rear panel is joined to at least one front panel 245 to define the body 220. The front panel 245 comprises a crotch panel 250 configured to cover the crotch and genitals of the wearer. In one embodiment, the crotch panel 250 comprises moisture impermeable material designed to prevent STDs that are transmitted through skin-to-skin contact from passing through the crotch panel 250. In one embodiment, the crotch panel 250 comprises latex material coupled with the front panel 245. In another embodiment, the crotch panel 250 is sewn to the front panel 245.

In another embodiment, the front panel 245 comprises moisture impermeable material designed to prevent STDs that are transmitted through skin-to-skin contact from passing through the front panel 245. In one embodiment, the front panel 245 comprises latex material coupled with the crotch panel 250.

In another embodiment, the rear panel comprises moisture impermeable material designed to prevent STDs that are transmitted through skin-to-skin contact from passing through the rear panel. In one embodiment, the rear panel comprises latex material coupled with the front panel 245.

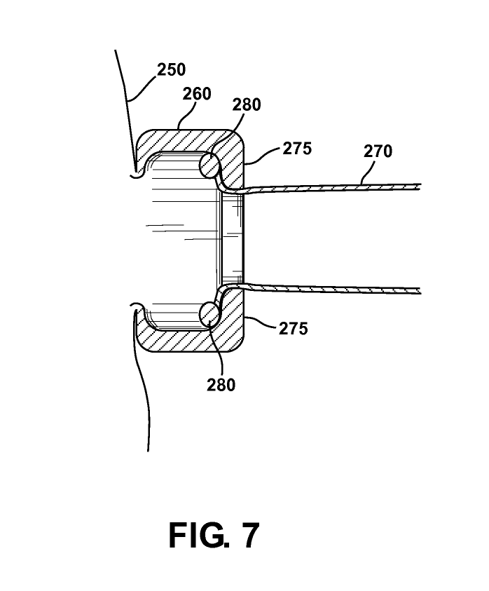

In one embodiment, the crotch panel 250 comprises a condom coupling member 260. The condom coupling member 260 defines an opening 265 sufficient to accommodate the wearer's penis. In one embodiment, the condom coupling member 260 comprises latex material. In another embodiment, the condom coupling member 260 is part of the crotch panel 250. In one embodiment, the condom coupling member 260 comprises an inverted outer edge 275 configured to accommodate a rubber band 280 associated with a condom 270, as shown in FIG. 7. The inverted outer edge 275 is configured to prevent the condom 270 from slipping from the condom coupling member 260.

Referring to FIGS. 6-7, before intercourse a user puts on the condom 270 followed by putting on the garment 210. Because the crotch panel 250 comprises moisture impermeable material, the crotch panel 250 protects the wearer's crotch area from STDs that are transmitted through skin-to-skin contact.

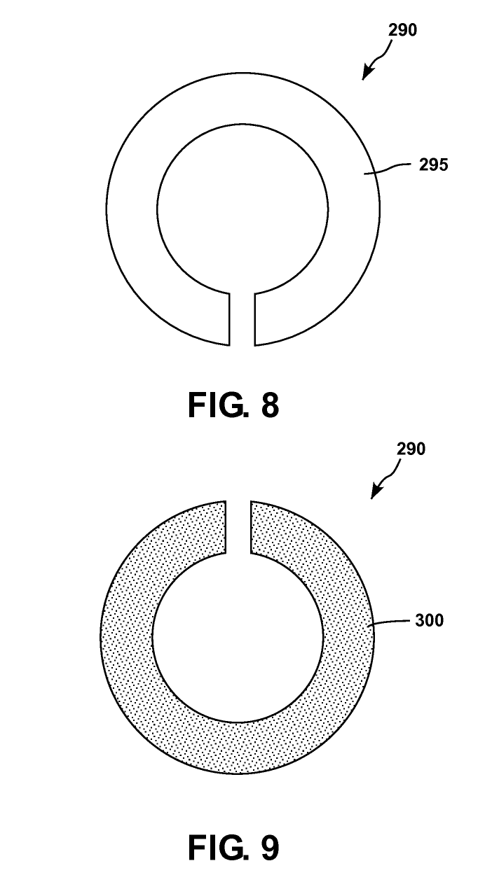

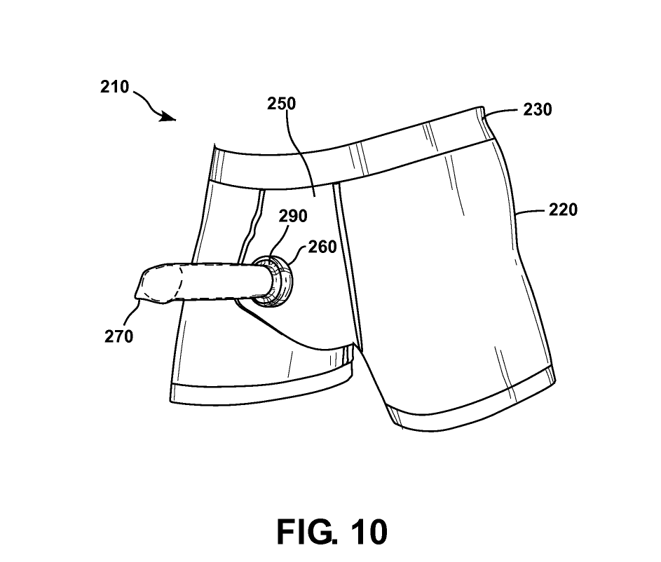

Referring to FIGS. 8-10, in another embodiment, a fastener 290 is applied to couple the condom 270 with the condom coupling member 260. In one embodiment, the fastener 290 is a flexible tape comprising an outer surface 295 and an adhesive surface 300 as shown in FIGS. 8-9. When applied, the adhesive surface 300 couples a portion of the condom 270 with a portion of the condom coupling member 260 as shown in FIG. 10. In one embodiment, the fastener 290 comprises latex material and adhesive.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

While several illustrative embodiments of the invention have been shown and described, numerous variations and alternative embodiments will occur to those skilled in the art. Such variations and alternative embodiments are contemplated, and can be made without departing from the scope of the invention as defined in the appended claims.

As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the claims. No claim element herein is to be construed under the provisions of 35 U.S.C. Sec. 112, sixth paragraph, unless the element is expressly recited using the phrase “means for . . .” and no method or process step herein is to be construed under those provisions unless the step, or steps, are expressly recited using the phrase “step(s) for . . . .”

What is claimed is:

1. A garment comprising:
a rear panel; and
a crotch panel coupled with the rear panel defining a condom coupling member, wherein the rear panel and the crotch panel form a first leg opening and a second leg opening, wherein the condom coupling member is configured to couple a condom with the crotch panel, wherein the crotch panel comprises material configured to prevent STDs that are transmitted through skin-to-skin contact from passing through the crotch panel.

2. The garment of claim 1, wherein the condom coupling member comprises an outer edge that defines a trench configured to accommodate a rubber band associated with the condom.

3. The garment of claim 1, wherein the condom coupling member comprises an inverted outer edge configured to accommodate a rubber band associated with a condom.

4. The garment of claim 1, further comprising a fastener configured to couple the condom with the condom coupling member.

5. The garment of claim 4, wherein the fastener comprises an adhesive surface for coupling the condom with the condom coupling member.

6. A method comprising:
providing a garment comprising:
a rear panel; and
a crotch panel couple with the rear panel defining a condom coupling member, wherein the rear panel and the crotch panel form a first leg opening and a second leg opening, wherein the crotch panel comprises material configured to prevent STDs that are transmitted through skin-to-skin contact from passing through the crotch panel;
providing a condom; and
coupling the condom with the condom coupling member.

7. The method of claim 6 further comprising:
providing a fastener; and
coupling the fastener with the condom and the condom coupling member.
